(19) United States
(12) Patent Application Publication
(10) Pub. No.: US 2015/0374266 A1
(43) Pub. Date: Dec. 31, 2015

(54) POSTURE DETECTION DEVICE

(71) Applicants: Oded COHEN, Savyon (IL); FUNTECH LTD., Zurich (CH)

(72) Inventors: Oded COHEN, Savyon (IL); Eliahu ELIACHAR, Haifa (IL); Nir LILACH, Kfar Yehoshua (IL)

(73) Assignee: UPRIGHT TECHNOLOGIES LTD., Tel Aviv (IL)

(21) Appl. No.: 14/766,614

(22) PCT Filed: Feb. 17, 2014
(86) PCT No.: PCT/IB2014/059041

§ 371 Date: Aug. 7, 2015

Related U.S. Application Data
(60) Provisional application No. 61/765,823, filed on Feb. 18, 2013.

(51) Int. Cl. A61B 5/11; A61B 5/00

(52) U.S. Cl. CPC A61B 5/1116; A61B 5/4566; A61B 5/746; A61B 5/684; A61B 5/6838; A61B 5/6831; A61B 5/0002; A61B 5/7405; A61B 2562/0219; A61B 2562/0261

(57) ABSTRACT

A device for detecting an unacceptable deviation from a neutral spine position in a user is provided. The device is configured to determine changes in orientation and curvature of the user's back, and comprises a first sensor configured to determine the change in orientation, a second sensor configured to determine the change in curvature, a signaling mechanism configured to issue an alert to the user, an attachment element configured to maintain the device on the user, and a controller configured to determine that an unacceptable deviation has occurred based on readings of the first and second sensors.

### FIELD OF THE INVENTION

The present disclosure relates to posture detection and feedback devices. In particular, the present disclosure relates to devices configured to detect deviations from good posture and to alert a user.

### BACKGROUND OF THE INVENTION

Good posture contributes towards a person's general wellbeing. By maintaining a good posture (sometimes referred to as the “neutral spine position”), a person can reduce fatigue, and lower the incidence pains, especially in the back, neck, and head, and help internal organs function with high efficiency. Good posture depends on both the orientation of a person's back, as well as its curvature.

Devices which are designed to detect poor posture and alert a user when such posture is detected are known in the art. In general, such devices are worn by a user, and comprise a sensor to determine the orientation of the user's back, and means for alerting the user when the reading of the sensor is consistent with poor posture.

### SUMMARY OF THE INVENTION

According to one aspect of the presently disclosed subject matter, there is provided a device for detecting an unacceptable deviation from a neutral spine position in a user, the device being configured to determine changes in orientation and curvature of the user's back, the device comprising:
a first sensor configured to determine the change in orientation, and a second sensor configured to determine the change in curvature;
a signaling mechanism configured to issue an alert to the user;
an attachment element configured to maintain the device on the user; and
a controller configured to determine that an unacceptable deviation has occurred based on readings of the first and second sensors.

The first sensor may comprise a tilt sensor. The tilt sensor may comprise an accelerometer.

The second sensor may comprise a strain gauge.

The controller may be configured, when it determines that the deviation (i.e., the unacceptable deviation) has occurred, to direct the signaling mechanism to issue the alert.

The device may further comprise a strain gauge assembly comprising the strain gauge and a carrying member curved toward a contact face thereof, wherein the strain gauge is mounted to the carrying member to detect mechanical strain therein. The device may be designed such that, when worn by the user, the contact face faces the user.

The device may further comprise a housing containing therewithin the tilt sensor, the housing being mounted to the strain gauge assembly on a side opposite the contact side.

The housing may be pivotally articulated to the strain gauge assembly.

The carrying member may be formed as a leaf spring. The leaf spring may be arranged such that, in a rest state thereof, it is convex in the direction opposite the contact face.

The controller may be configured to execute a subroutine to facilitate the user to position the device on his back.

The subroutine may comprise determining, using the first sensor, the vertical orientation of the device, and indicating to the user when the device is in a vertical position.

The subroutine may comprise determining, using the first sensor, the vertical orientation of the device, and indicating to the user only when the device is not in a vertical position. The controller may be configured to execute the subroutine when the device is tilted within a predefined range of angles.

The subroutine may comprise communicating to a remote computing device the vertical orientation of the device.

The attachment element may comprise a first component of a hook and loop fastener. The first component may comprise the hooks of the hook and loops fastener. The device may further comprise strips having a first face comprising a second component of the hook and loop fastener, and a second face comprising an adhesive suitable for removably securing to the user's skin.

The attachment element may comprise a clip.

The attachment element may be is configured for attached to one or more straps. The attachment element may comprise the straps.

The alert may comprise any one or more of a vibration, an audible sound, and communication with a remote computing device.

According to an optional modification of the above aspect, the device may comprise only a sensor configured to determine the change in curvature, mutatis mutandis.

### BRIEF DESCRIPTION OF THE DRAWINGS

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. In the accompanying drawings:

FIG. 1A is a perspective view of a device according to the presently disclosed subject matter;

FIG. 1B illustrates an area of placement of the device on a user;

FIGS. 2A and 2B are, respectively, perspective and side views of a strain gauge assembly of the device illustrated in FIG. 1A;

FIG. 3 is a strip for use with the device illustrated in FIG. 1A;

FIG. 4 is an exploded view of a housing of the device illustrated in FIG. 1A;

FIG. 5A is a bottom perspective view of a base of the housing illustrated in FIG. 4;

FIG. 5B is a cross-section taken along line V-V in FIG. 5A;

FIG. 6 illustrates a deviation of the device illustrated in FIG. 1A from the vertical; and

FIG. 7 illustrates a remote computing device used in a positioning routine of the device illustrated in FIG. 1A.

### DETAILED DESCRIPTION OF EMBODIMENTS

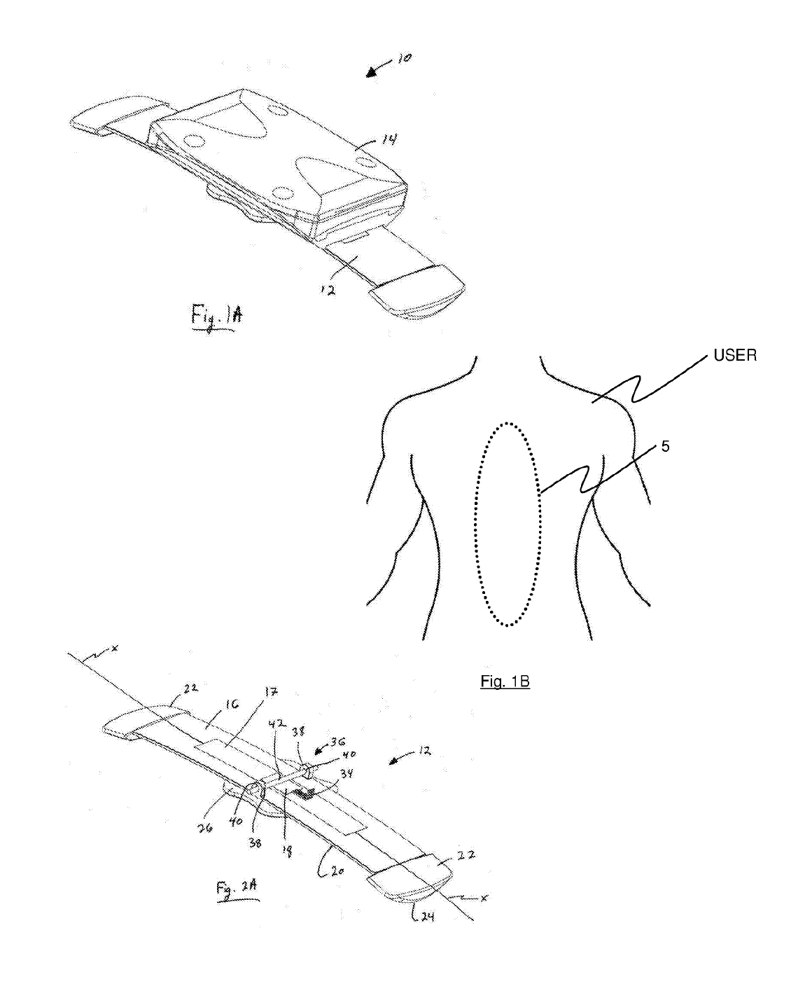

As illustrated in FIG. 1A, there is provided device, which is generally indicated at 10, for detecting an unacceptable deviation from a neutral spine position undergone by a user wearing it. It will be appreciated that the term “neutral spine position” as used herein refers to generally good posture of a human user, which may include deviations from a strict medical definition of the neutral spine position, and that an unacceptable deviation therefrom includes posture generally considered to be undesirable, such as slouching, etc. (as opposed to deviations therefrom which are not considered to be undesirable, such as bending down, etc.). It is further configured to alert the user when it detects that a deviation has occurred. (Herein the specification and claims, unless otherwise stated, references to a “deviation” refer to an unacceptable deviation from a neutral spine position.)

The device 10 is configured to be worn by the user, for example as illustrated in FIG. 1B on his back (in the area indicated at 5) or his chest area, and is designed to detect both the orientation and curvature of his back. In addition, it may also be worn on a user's chest, or on any other suitable area. As such, it comprises a sensor configured to determine the orientation of a user's back, and a sensor configured to determine changes in curvature of a user's back. Using the feedback from these sensors, the device 10 is configured to determine whether a deviation has occurred.

The device 10 comprises a strain gauge assembly 12, which is configured to facilitate detecting the curvature of the user's back, and a housing 14, which contains, inter alia, elements configured to detect the orientation of the user's back. The device 10 is designed to be worn by the user such that the strain gauge assembly 12 is adjacent the user, with the housing 14 being disposed outwardly.

As will be described below, the strain gauge assembly 12 and housing are pivotally articulated to one another, so as to facilitate detecting the curvature of the user's back while minimizing the effect the curvature has on detection of the orientation.

As illustrated in FIGS. 2A and 2B, the strain gauge assembly 12 comprises a carrying member 16 with a strain gauge 18 mounted thereon. The carrying member 16 is formed as a flexible sheet, constituting a leaf spring, convex in the direction of the housing 14 (i.e., it is curved away from the housing and toward the user when wearing the device 10). The carrying member 16 may be made of any suitable material. If it is formed so as to be unsuitable for use with the strain gauge 18, e.g., owing to its material and/or construction, a secondary carrier 17, which may be made, e.g., of a metal, may be mounted thereon, with the strain gauge 18 mounted thereto.

The side of the carrying member 16 facing the direction in which it is curved constitutes a contact face 20 thereof, which is designed to face toward the user during use. Terminal caps 22 are provided at either end of the carrying member 16, each having a projection 24 formed in the direction of curvature of the carrying member 16 (i.e., toward the user). The terminal caps 22 are configured to facilitate sliding of the ends of the carrying member 16 along the user's back. It made of a smooth material, such as a suitable plastic, contributing towards the comfort of the device 10 when being worn by a user.

The carrying member 16 further comprises, on the contact face 20 thereof, an attachment element 26. According to some example, the attachment element 26 comprises a first component of a hook and loop fastener, for example as sold under the trade name VELCRO™. As illustrated in FIG. 3, a replaceable strip 28 is provided to facilitate securing the device 10 to a user utilizing the attachment element as illustrated in FIGS. 2A and 2B. The strip 28 comprises a first face 30 with the second component of the hook and loop fastener corresponding to the first component thereof provided on the attachment element 26, and a second face 32 comprising an adhesive suitable for removably securing to the user's skin. According to some modifications, the second component (provided on the strip 28) is the loop component of the hook and loop fastener, which is typically the less expensive one to provide, and the attachment element comprises the hook fastener, which may be, e.g., #7335 Low-Profile Polyolefin Hook Fastener as provided by The 3M Company™. Thus, a user may purchase a package of strips, each to be used once (or a small number of times).

According to other examples (not illustrated), an attachment element may be provided, either in addition to or instead of the attachment element 26 described above with reference to FIGS. 2A and 2B, comprising a clip, thereby facilitating the device to be attached to an article of clothing worn by the user (e.g., a waistband, a strap of a brassiere, etc.), or one or more straps, enabling the user to wear the device 10 strapped around his body. The clip may be formed on the side of the housing 14 opposite the contact face 20 of the carrying member, facilitating attachment of the device between the article of clothing and skin of the user. The clip may be spring loaded, or defined by formation of a slot into which the article of clothing is inserted. The device 10 may further be provided with a strap (not illustrated) configured to be worn around the user's torso, and received within the clip.

The strain gauge 18 may be any suitable device configured to detect and measure the strain undergone by the carrying member 16. It may be oriented so as to be more sensitive to detecting strain along the longitudinal axis X of the carrying member 16. The strain gauge 18 may comprise a Wheatstone bridge to determine the change in electrical resistance thereof, as is well known in the art, thereby determining the strain. Reverting to FIGS. 2A and 2B, electrical leads 34 are provided to connect the strain gauge 18 to other components of the device 10, as will be described below.

The carrying member 16 may be provided with a pivot mechanism, which is generally indicated at 36, configured to pivotally articulate it to the housing 14, as mentioned above. The pivot mechanism 36 comprises a pair of mounts 38 protruding from edges of the carrying member 16, at or near the center of its length, away from the contact face 20. Each of the mounts 38 comprises an aperture 40, which may be through-going (as illustrated) or blind. An axle 42 is provided, spanning between the mounts 38 and received within the apertures 40. The axle may be fixed therein such that its rotation is prevented.

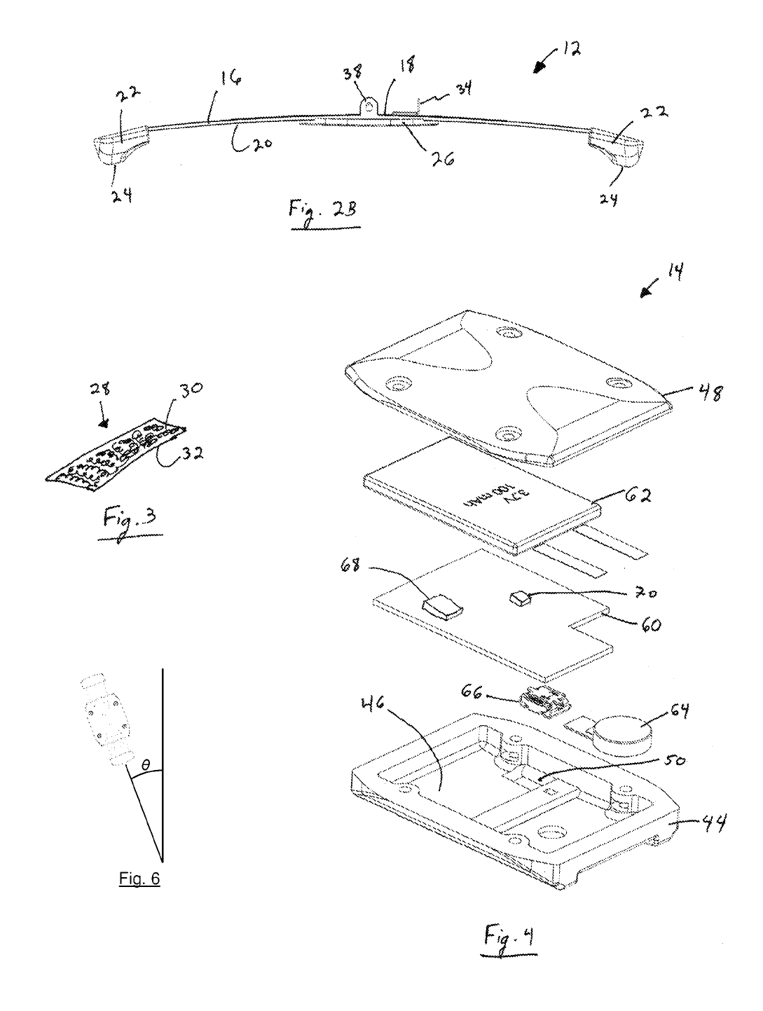

The housing 14 comprises other functional elements of the device 10. As illustrated in FIG. 4, the housing 14 comprises a base 44 defining a chamber 46, and a cover 48 mounted thereto. A recess 50, having one side open to the outside of the base 44 (best seen in FIG. 5A), is formed within the chamber 46, the purpose of which will be described below.

As illustrated in FIGS. 5A and 5B, the bottom (i.e., strain gauge assembly-facing) side of the base 44 comprises a through-going aperture 52 to allow passage therethrough of the electrical leads 34 associated with the strain gauge 18. In addition, it is formed with a pair of sockets 54, each configured to receive therein one of the mounts 38 of the pivot mechanism 36 of the carrying member 16. The sockets 54 are formed slightly larger than the mounts 38, so as to allow them to pivot therewithin. As best seen in FIG. 5B, a through-going bore 56 is formed in the base 44, to accommodate therein the axle 42 of the pivot mechanism 36.

The bottom side of the base 44 may comprise a pair of curved rockers 58, configured to accommodate pivoting of the housing 14 with respect to the strain gauge assembly 12.

Reverting to FIG. 4, provided within the cavity 46 of the base 44 are a printed circuit board (PCB) 60, battery 62, and a vibrator 64. In addition, an electrical connector 66 is provided within the recess 50, with one side thereof exposed to the outside of the housing 14 via the open side thereof.

The PCB 60 comprises functional elements of the device 10, and is configured to direct its operation. As such, it comprises a controller 68, which may comprise a central processing unit having suitable elements for controlling the device, including, but not limited to, one or more microprocessors, volatile and/or non-volatile memory, etc. In addition, it comprises a tilt sensor 70, such as an accelerometer, which is configured for determining the orientation of the device. The PCB 60 is connected to the electrical leads 34 associated with the strain gauge 18, in order to receive input therefrom. Electrical power for the PCB 60 and its components is provided by the battery 62.

Electrical communication between the controller 68 and external data/power sources may be provided via the electrical connector 66. Accordingly, it may comprises a receptacle (not seen) configured for receiving therein a data and/or power plug, for example as defined by one or more of the USB standards. The device 10 may be configured to transfer data to and from the controller 68, such as usage history, software updates, etc. In addition, the electrical connector 66 facilitates supplying power to the device, e.g., to charge the battery 62, etc.

It will be appreciated that data transfer and/or power supply may be accomplished by wired means, for example wireless data transmission and inductive power transfer, obviating the need for the electrical connector 66.

The vibrator 64 constitutes a signaling mechanism, and is thus configured for producing a silent alert to the user. It may comprise, e.g., an electric motor connected to an eccentric weight. According to some examples, the signaling mechanism may comprise software loaded on the controller 68 configured to communicate with a remote computing device, such as a smartphone, via any suitable wireless technology. The smartphone is loaded with software configured to alert the user based on the communication. According to other examples, the signaling mechanism may comprise an audible alert.

In use, the controller 68 is configured to determine whether a deviation has occurred, and whether or not to alert the user based on this determination. The user wears the device 10 on his back in a vertical position. His back bears against the terminal caps 22 of the carrying member 16, causing it to bend. This change in shape of the carrying member 16 is measured by the strain gauge 18. Changes in the orientation of the user's back are detected and measured by the tilt sensor 70. Changes in the curvature of the user's back (which may indicate, e.g., slouching) cause the back to bear on the terminal caps 22 less, thereby changing the shape of the carrying member 16, which is detected and measured by the strain gauge 18. The controller 68 uses the readings from both the strain gauge 18 and the tilt sensor 70 to determine whether or not an unacceptable deviation has occurred.

By accounting for readings from both the strain gauge 18 and the tilt sensor 70, the controller 68 may reduce the incidence of false positives (i.e., determining that a deviation has occurred when none has, or when it is acceptable) compared to if it were to account for readings from only one. Thus, the combination of the strain gauge 18 and the tilt sensor 70 may increase the accuracy of the device 10, for example when the user is moving. When the controller 68 determines that a deviation has occurred, it may issue a command to the signaling mechanism (e.g., the vibrator 64) to alert the user, so that he may correct his posture.

The controller 68 is configured with software to perform one or more actions to facilitate the detection. For example, it may be configured to determine, based on readings obtained from the strain gauge 18 and the tilt sensor 70, that a deviation has occurred. For example, in a case wherein the strain gauge 18 indicates that the curvature or the user's back is consistent with a deviation, but the tilt sensor 70 indicates that the orientation of the user's back changed rapidly, the controller 68 may determine that an acceptable deviation has occurred, for example that the user bent down or crouched intentionally. Additionally, the tilt sensor 70 may indicate that the orientation of the back is consistent with a neutral spine position, but the strain gauge 18 may indicate a change in curvature. In such a case, the controller 68 may be configured to determine, e.g., based on the magnitude and rate of change of the curvature of the user's back, whether an unacceptable deviation has occurred; for example, it may determine that an unacceptable deviation of hunched shoulders has occurred.

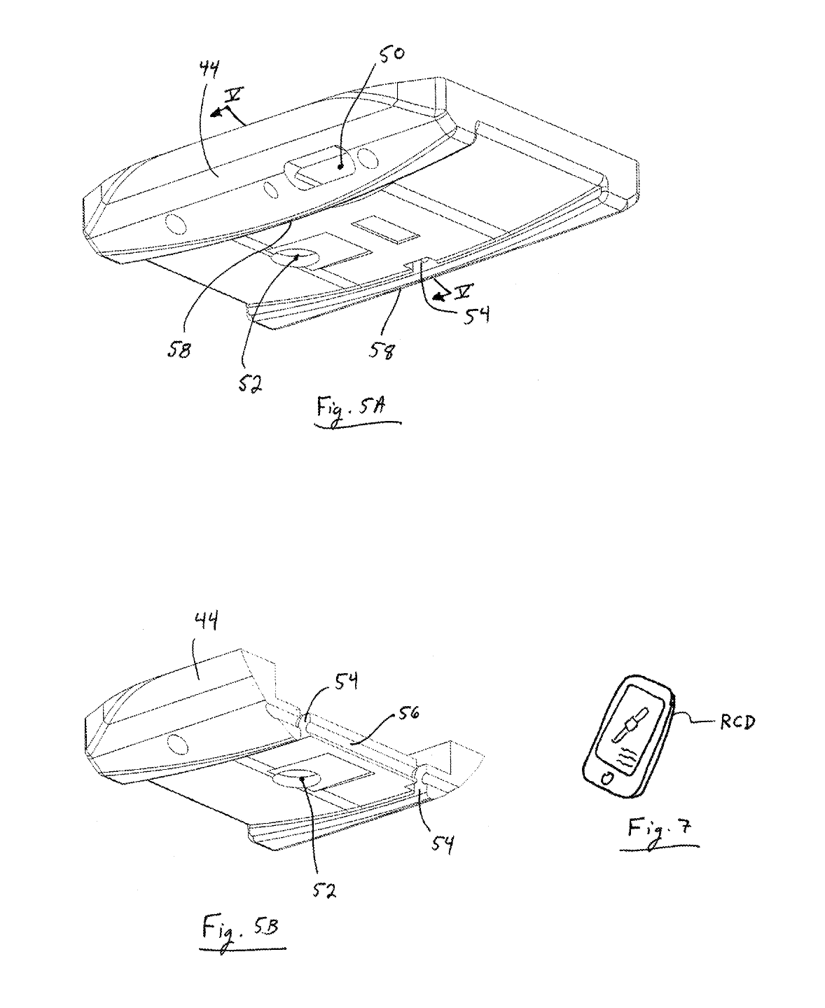

In addition, the controller 68 may be configured to execute a positioning subroutine, wherein it assists the user to position the device 10 on his back without being able to view it during placement. According to one example, when executing the positioning subroutine, the controller 68 is configured to issue an alert to the user when the device 10 is disposed vertically. According to another example, the controller 68 is configured to issue an alert when the device 10 is not disposed vertically, i.e., when it needs repositioning. According to the latter example, the positioning subroutine may include a restriction that the controller 68 only issue the alert when the deviation from a vertical position of the device 10 exceeds a predetermined range, for example if the deviation from the vertical exceeds an angle θ, for example as illustrated in FIG. 6.

According to other examples, which may supplement or supplant the examples of positioning subroutines disclosed above, the positioning subroutine includes the controller 68 communicating with a remote computing device (RCD), such as a smartphone, regarding the position of the device 10, which the RCD may communicate the user, for example as illustrated in FIG. 7.

It will be appreciated that the device 10 disclosed above may be configured to monitor other aspects of a user's activities, including, but not limited to, breathing parameters (e.g., rate depth, etc.), walking gait, etc., without departing from the scope of the present disclosure, mutatis mutandis.

Those skilled in the art to which this invention pertains will readily appreciate that numerous changes, variations and modifications can be made without departing from the scope of the invention mutatis mutandis.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the disclosure.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

1. A device for detecting an unacceptable deviation from a neutral spine position in a user, the device being configured to determine changes in orientation and curvature of the user's back, the device comprising:
a first sensor configured to determine the change in orientation, and a second sensor configured to determine the change in curvature;
a signaling mechanism configured to issue an alert to the user;
an attachment element configured to maintain the device on the user; and
a controller configured to determine that an unacceptable deviation has occurred based on readings of said first and second sensors.

2. The device according to claim 1, wherein said first sensor comprises a tilt sensor.

3. The device according to claim 2, wherein said tilt sensor comprises an accelerometer.

4. The device according to any one of the preceding claims, wherein said second sensor comprises a strain gauge.

5. The device according to any one of the preceding claims, wherein said controller is configured, when it determines that said deviation has occurred, to direct said signaling mechanism to issue said alert.

6. The device according to any one of claims 4 and 5, further comprising a strain gauge assembly comprising said strain gauge and a carrying member curved toward a contact face thereof, wherein said strain gauge is mounted to said carrying member to detect mechanical strain therein.

7. The device according to claim 6, designed such that, when worn by the user, said contact face faces the user.

8. The device according to any one of claims 6 and 7, further comprising a housing containing therewithin said tilt sensor, said housing being mounted to said strain gauge assembly on a side opposite said contact side.

9. The device according to claim 8, wherein said housing is pivotally articulated to said strain gauge assembly.

10. The device according to any one of claims 6 through 9, wherein said carrying member is formed as a leaf spring.

11. The device according to claim 10, wherein said leaf spring is arranged such that, in a rest state thereof, it is convex in the direction opposite said contact face.

12. The device according to any one of the preceding claims, wherein said controller is configured to execute a subroutine to facilitate the user to position the device on his back.

13. The device according to claim 12, wherein said subroutine comprises determining, using said first sensor, the vertical orientation of the device, and indicating to the user when the device is in a vertical position.

14. The device according to claim 12, wherein said subroutine comprises determining, using said first sensor, the vertical orientation of the device, and indicating to the user only when the device is not in a vertical position.

15. The device according to claim 14, wherein said controller is configured to execute said subroutine when the device is tilted within a predefined range of angles.

16. The device according to any one of claims 12 through 15, wherein said subroutine comprises communicating to a remote computing device the vertical orientation of the device.

17. The device according to any one of the preceding claims, wherein said attachment element comprises a first component of a hook and loop fastener.

18. The device according to claim 17, wherein said first component comprises the hooks of the hook and loops fastener.

19. The device according to any one of claims 17 and 18, further comprising strips having a first face comprising a second component of said hook and loop fastener, and a second face comprising an adhesive suitable for removeably securing to the user's skin.

20. The device according to any one of claims 1 through 16, wherein said attachment element comprises a clip.

21. The device according to any one of claims 1 through 16, wherein said attachment element is configured for being attached to one or more straps.

22. The device according to claim 21, wherein said attachment element comprises said straps.

23. The device according to any one of the preceding claims, wherein said alert comprises a vibration.

24. The device according to any one of the preceding claims, wherein said alert comprises an audible sound.

25. The device according to any one of the preceding claims, wherein said alert comprises communication with a remote computing device.

26. A device substantially as described herein and illustrated in the accompanying drawings.
